(19) United States
(12) Patent Application Publication
(10) Pub. No.: US 2024/0246903 A1
(43) Pub. Date: Jul. 25, 2024

(54) INDANONE AND TETRALONEKETO OR HYDROXYL OXIMES AS CANCEL THERAPEUTICS

(71) Applicant: Board of Regents, The University of Texas System, Austin, TX (US)

(72) Inventors: Stanton McHardy, San Antonio, TX (US); Michael W. Tidwell, San Antonio, TX (US); Andrew J. Brenner, San Antonio, TX (US); Ratna K. Vadlamudi, San Antonio, TX (US)

(21) Appl. No.: 18/558,765

(22) PCT Filed: Apr. 28, 2022
(86) PCT No.: PCT/US2022/026846

§ 371 Date: Nov. 3, 2023

Related U.S. Application Data
(60) Provisional application No. 63/183,764, filed on May 4, 2021.

(51) Int. Cl. C07C 251/44; A61P 35/00; C07C 311/16; C07C 311/32; C07D 231/56; C07D 239/34; C07D 257/04; C07D 317/50

(52) U.S. Cl. CPC C07C 251/44; A61P 35/00; C07C 311/16; C07C 311/32; C07D 231/56; C07D 239/34; C07D 257/04; C07D 317/50; C07C 2602/08; C07C 2602/10

(57) ABSTRACT

Certain embodiments are directed to compounds, pharmaceutically acceptable salts, stereoisomers and prodrugs thereof, that are ER ligands and particularly to such compounds that are ERβ selective and/or ERβ specific ligands.

### RELATED APPLICATIONS

This application claims priority to U.S. Provisional Application Ser. No. 63/183,764 filed May 4, 2021, which is incorporated herein by reference in its entirety.

### STATEMENT REGARDING FEDERALLY FUNDED RESEARCH

This invention was made with government support under CA178499 and P30CA054174-17 awarded by the National Institutes of Health. The government has certain rights in the invention.

### BACKGROUND

This invention relates generally to estrogen receptor (ER) ligands, and particularly to ligands that exhibit subtype selective differences in ligand binding, transcriptional potency or efficacy for ER beta (ERβ).

The estrogen receptor, a member of the nuclear hormone receptor superfamily, mediates the activity of estrogens in the regulation of a number of important physiological processes, including the development and function of the female reproductive system and the maintenance of bone density and cardiovascular health. A variety of estrogen pharmaceuticals have been developed to regulate these processes or their pathological counterparts, including infertility, breast cancer, and osteoporosis.

ER is a transcription factor that binds to specific estrogen response elements (ERE) in the promoter region of estrogen-regulated genes and whose activity for transcription is modulated by the estrogen ligands (Katzenellenbogen and Katzenellenbogen, (1996) Chem. Biol., 3:529-536). The capacity of ER-ligand complexes to activate gene transcription is mediated by a series of co-regulator proteins (Horwitz et al. (1996) Mol. Endocrinol., 10:1167-1177). These co-regulators have interaction functions that tether ER to the RNA polymerase pre-initiation complex, as well as enzymatic activities to modify chromatin structure (Glass et al. (1997) Curr. Opin. Cell. Biol., 9:222-232).

The differential responses observed have raised the interesting issue of tissue-, cell-, and gene-specific activity of estrogens which is based on the ligand, the receptor, and/or the effector site and has been termed “tripartite receptor pharmacology”. (Katzenellenbogen et al, (1996) Mol. Endocrinol. 10, 119-131.) Each cell type and each gene presents to an ER(subtype)-ligand complex a unique combination of these effector components—various estrogen response elements and co-regulators—that appear to underlie, in part, the cell and gene selectivity of various estrogens. Extensive efforts are being expended to develop ligands which selectively antagonize or agonize estrogenic effects.

It had been assumed that estrogen-related events were mediated by only one estrogen receptor. However, the discovery of a second estrogen receptor (ERβ) (Mosselman et al., (1996) FEBS Lett., 392, 49-53; Kuiper et al., (1996) Proc. Natl. Acad. Sci. USA, 93, 5925-5930) indicates that tissue- and cell-selectivity of certain estrogens may be due, in part, to their mediation through ERβ separate from, or in conjunction with, the classical estrogen receptor (ERα). This possibility has been supported by the difference in tissue distribution between ERα and ERβ (Mosselman et al., (1996) FEBS Left. supra; Kuiper et al., (1997) Endocrinology 138:863-870; Saunders et al., (1997) J. Endocrinol. 154:R13-R16; Register and Adams, (1998) J. Steroid Biochem. Mol. Biol. 64:187-191.)

ERα and ERβ exhibit complex tissue distributions. Certain tissues may contain only (or predominately) ERα or ERβ and other tissues may contain a mixture of both. Tissues that exhibit high levels of ERβ include, for example, prostate, testes, ovaries, gastrointestinal tract, lung, bladder, hematopoetic and central nervous systems, and certain regions of the brain, whereas ERα predominates in the uterus, breast, kidney, liver and heart. Many tissues contain both ERα and ERβ, such as breast, epididymis, thyroid, adrenal, bone, and certain other regions of the brain. Furthermore, it has been shown that the pharmacology of traditional ER agonists and antagonists is reversed for ERβ in the context of certain ER effector sites. (Paech, K. et al. (1997) Science 277:1508-1510.)

Other ERβ agonists have been developed but suffer from either limited efficacy due to inadequate potency, inadequate selectivity for the beta isoform [ESR2 v ESR1], poor penetration into the brain, or a combination of these. Additionally, of those that have penetration into the brain, none are in clinical development or commercially available.

Thus, there remains a need for additional ERβ agonist compositions.

### SUMMARY

In addressing the need for additional ERβ agonist a solution has been discovered in the form of new molecules that are effective ERβ agonist that can be used as therapeutic agents.

Certain embodiments are directed to compounds, pharmaceutically acceptable salts, stereoisomers and prodrugs thereof, that are ER ligands and particularly to such compounds that are ERβ selective and/or ERβ specific ligands. In certain embodiments, the invention relates to compounds which are ERβ selective agonists. In specific embodiments, the invention relates to compounds, pharmaceutically acceptable salts, stereoisomers, and prodrugs thereof which are ERβ selective agonists and which exhibit minimal agonist or antagonist effect on ERα. Certain drug candidates have improved potency with effects in the 2-3 μM range, which is a 4 fold improvement over existing agonists. Certain embodiments also provide improved selectivity over ERα.

Certain embodiments are directed to compounds of Formula I:

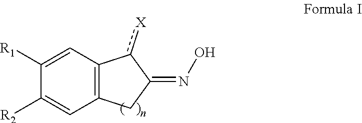

wherein:
R₁ and R₂ are independently selected from hydrogen, alkyl, branched alkyl, substituted alkyl, aryl (Ar), substituted Ar, —CH₂—Ar, and —CH₂-substituted AR. The aryl can be a fused aryl, heterocyclic, or heteroaryl, optionally substituted with one or more R₃ groups. R₃ is selected from halogen (F, Cl, Br and I), —OH, —O—R₄, —NO₂, CO₂H, —CN, —CF₃, CO₂—R₄, CONH₂, CONHR₄, CON(R₄)₂, —NH₂, —NHR₄, —N(R₄)₂, —NHC(O)R₄, alkyl, branched alkyl, substituted alkyl, halogenated methyl (—CF₃, —CHF₂ and the like), and halogenated O-alkyl (i.e. —OCF₃). R₄ is selected from branched or unbranched alkyl, halogenated alkyl, or heteroaryl. Unless otherwise indicated, as used herein, the term “aryl” includes an organic radical derived from an aromatic hydrocarbon by removal of one hydrogen, such as phenyl (Ph), naphthyl, indenyl, indanyl and fluorenyl. “Aryl” encompasses fused ring groups wherein at least one ring is aromatic. Unless otherwise indicated, as used herein, “heteroaryl” refers to aromatic groups containing one or more heteroatoms, preferably from one to three heteroatoms, selected from O, S and N. A multicyclic group containing one or more heteroatoms wherein at least one ring of the group is aromatic is a “heteroaryl” group. X is selected from O, —OH, N—OH, H₂, and —O—R₅, bonded by a single bond or double bond as depicted. R₅ is selected from branched or unbranched alkyl, halogenated alkyl, heteroaryl, —C(O)—R₄, and —NH—C(O)—R₄, wherein R₄ is as above, selected from branched or unbranched alkyl, halogenated alkyl, or heteroaryl. And n is 1 or 2.

Certain aspects are directed to isomers of Formula I or Formula II. In particular E, Z, or E and Z oxime or keto-oxime isomers. Isotope analogs of Formula I or Formula II are also contemplated, e.g., deuterium analogs, as well as other isotopes.

Certain embodiments are directed to compounds having a IUPAC name of (2Z)-6-(3-chloro-4-hydroxyphenyl)-2-(hydroxyimino)-2,3-dihydro-1H-inden-1-one; (2Z)-6-[4-hydroxy-3-(trifluoromethyl)phenyl]-2-(hydroxyimino)-2,3-dihydro-1H-inden-1-one; 2-hydroxy-5-[(2Z)-2-(hydroxyimino)-3-oxo-2,3-dihydro-1H-inden-5-yl]benzonitrile; (2Z)-2-(hydroxyimino)-6-(4-hydroxyphenyl)-2,3-dihydro-1H-inden-1-one; (2Z)-2-(hydroxyimino)-6-(4-hydroxyphenyl)-2,3-dihydro-1H-inden-1-one; (2Z)-2-(hydroxyimino)-6-(1-methyl-1H-indazol-6-yl)-2,3-dihydro-1H-inden-1-one; (2Z)-6-(3-chloro-4-hydroxyphenyl)-2-(hydroxyimino)-2,3-dihydro-1H-inden-1-one; (2Z)-6-(3-chloro-4-hydroxyphenyl)-2-(hydroxyimino)-2,3-dihydro-1H-inden-1-one; 4-[(2Z)-2-(hydroxyimino)-2,3-dihydro-1H-inden-5-yl]-2-(trifluoromethyl)phenol; 2-chloro-4-[(2Z)-2-(hydroxyimino)-2,3-dihydro-1H-inden-5-yl]phenol; (2Z)-6-(3-fluoro-5-hydroxyphenyl)-2-(hydroxyimino)-2,3-dihydro-1H-inden-1-one; (2E)-6-(3-fluoro-4-hydroxyphenyl)-2-(hydroxyimino)-2,3-dihydro-1H-inden-1-ol; 2-fluoro-4-[3-(hydroxyimino)-2,3-dihydro-1H-inden-5-yl]phenol; 2-fluoro-4-[(3E)-3-(hydroxyimino)-2,3-dihydro-1H-inden-5-yl]phenol; (2Z)-2-(hydroxyimino)-6-(1H-indazol-6-yl)-2,3-dihydro-1H-inden-1-one; 2-fluoro-4-[(2Z)-2-(hydroxyimino)-2,3-dihydro-1H-inden-5-yl]phenol; 2-hydroxy-5-[(2E)-2-(hydroxyimino)-1-oxo-2,3-dihydro-1H-inden-5-yl]benzonitrile; (2E)-2-(hydroxyimino)-5-(4-hydroxyphenyl)-2,3-dihydro-1H-inden-1-one; (2E)-5-(3-fluoro-4-hydroxyphenyl)-2-(hydroxyimino)-2,3-dihydro-1H-inden-1-one; (2E)-5-(3-chloro-4-hydroxyphenyl)-2-(hydroxyimino)-2,3-dihydro-1H-inden-1-one; (2E)-5-[4-hydroxy-3-(trifluoromethyl)phenyl]-2-(hydroxyimino)-2,3-dihydro-1H-inden-1-one; (2E)-5-(3-fluoro-5-hydroxyphenyl)-2-(hydroxyimino)-2,3-dihydro-1H-inden-1-one; 4-[(1E)-1-(hydroxyimino)-2,3-dihydro-1H-inden-5-yl]-2-(trifluoromethyl)phenol; (2E)-5-(3-fluoro-5-hydroxyphenyl)-2-(hydroxyimino)-2,3-dihydro-1H-inden-1-ol; (2E)-6-(3-chloro-4-hydroxyphenyl)-2-(hydroxyimino)-1,2,3,4-tetrahydronaphthalen-1-one; 2-chloro-4-[(5E)-5-(hydroxyimino)-5,6,7,8-tetrahydronaphthalen-2-yl]phenol; (2Z)-6-(4-hydroxy-3-methylphenyl)-2-(hydroxyimino)-2,3-dihydro-1H-inden-1-one; (2Z)-6-(4-hydroxy-3-methoxyphenyl)-2-(hydroxyimino)-2,3-dihydro-1H-inden-1-one; (2Z)-6-(4-hydroxy-3,5-dimethylphenyl)-2-(hydroxyimino)-2,3-dihydro-1H-inden-1-one; (2Z)-6-(2H-1,3-benzodioxol-5-yl)-2-(hydroxyimino)-2,3-dihydro-1H-inden-1-one; (2Z)-2-(hydroxyimino)-6-(3-hydroxyphenyl)-2,3-dihydro-1H-inden-1-one; (2E)-2-(hydroxyimino)-6-[4-(2H-1,2,3,4-tetrazol-5-yl)phenyl]-2,3-dihydro-1H-inden-1-one; (2Z)-6-[4-hydroxy-2-(trifluoromethyl)phenyl]-2-(hydroxyimino)-2,3-dihydro-1H-inden-1-one; (2Z)-6-(2-chloro-4-hydroxyphenyl)-2-(hydroxyimino)-2,3-dihydro-1H-inden-1-one; (2Z)-6-(4-hydroxy-2-methylphenyl)-2-(hydroxyimino)-2,3-dihydro-1H-inden-1-one; (2Z)-2-(hydroxyimino)-6-(2-methoxypyrimidin-5-yl)-2,3-dihydro-1H-inden-1-one; (2Z)-6-(2,4-dimethoxyphenyl)-2-(hydroxyimino)-2,3-dihydro-1H-inden-1-one; (2Z)-6-(2-fluoro-4-hydroxyphenyl)-2-(hydroxyimino)-2,3-dihydro-1H-inden-1-one; (2Z)-6-(2,3-difluoro-4-hydroxyphenyl)-2-(hydroxyimino)-2,3-dihydro-1H-inden-1-one; (2Z)-6-(4-hydroxy-2-methylphenyl)-2-(hydroxyimino)-1,2,3,4-tetrahydronaphthalen-1-one; 4-[(5E)-5-(hydroxyimino)-5,6,7,8-tetrahydronaphthalen-2-yl]-3-methylphenol; or (2Z)-2-(hydroxyimino)-6-(2-hydroxypyrimidin-5-yl)-2,3-dihydro-1H-inden-1-one.

Certain embodiments are directed to the compounds CIDD-0150184 or (2E)-5-(2-ethenyl-4-hydroxyphenyl)-2-(hydroxyimino)-2,3-dihydro-1H-inden-1-one, and CIDD-0149897 or (2Z)-6-(3-fluoro-4-hydroxyphenyl)-2-(hydroxyimino)-2,3-dihydro-1H-inden-1-one.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention.

Throughout this application, the term “about” is used to indicate that a value includes the inherent variation of error for the device, the method being employed to determine the value, or the variation that exists among the study subjects.

he terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “IC₅₀” refers to an inhibitory dose that results in 50% of the maximum response obtained.

The term half maximal effective concentration (EC₅₀) refers to the concentration of a drug that presents a response halfway between the baseline and maximum after some specified exposure time.

The terms “inhibiting,” “reducing,” or “prevention,” or any variation of these terms, when used in the claims and/or the specification includes any measurable decrease or complete inhibition to achieve a desired result.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, infants and fetuses.

The use of the word “a” or “an” when used in conjunction with the term “comprising” in the claims and/or the specification may mean “one,” but it is also consistent with the meaning of “one or more,” “at least one,” and “one or more than one.”

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Effect of new ERβ agonists on activation of ERα vs ERβ activity measured using ERE reporter gene assays.

FIG. 2. Effect of new ERβ agonists on potentiation of ERβ activity in reporter gene assays.

FIG. 3. Effect of test compounds on reducing the cells viability of GBM cells.

FIG. 4. IC50 values of new ERβ agonists determined using Polar Screen Nuclear Receptor (NR) Competitive Binding Assay.

FIG. 5. Effect of new ERβ agonists on the cell viability of U251 and U252 ERβ-Ko cells.

FIG. 6. Effect of new ERβ agonist on the cell viability.

### DESCRIPTION

Certain embodiments are directed to indanone and tetralone-keto or hydroxyl oximes as useful therapeutics for the treatment of estrogen receptor beta (ERβ) expressing tumors (brain, breast, ovarian, prostate, salivary, etc) as well as for neuroprotection in conditions such as stroke and traumatic brain injury (TBI). These compounds interact and agonize ERβ, which has been shown to suppress tumor growth, sensitize tumors to chemotherapy, promote synaptic strength, neuroplasticity, and neurogenesis.

The term “effective amount” means an amount effective, at dosages and for periods of time necessary, to achieve the desired therapeutic or prophylactic result.

An “effective amount” of an anti-cancer agent in reference to decreasing cancer cell growth, means an amount capable of decreasing, to some extent, the growth of some cancer or tumor cells. The term includes an amount capable of invoking a growth inhibitory, cytostatic and/or cytotoxic effect and/or apoptosis of the cancer or tumor cells.

A “therapeutically effective amount” in reference to the treatment of cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of cancer or tumor cell infiltration into peripheral organs; (5) inhibition (i.e., reduction, slowing down, or complete stopping) of metastasis; (6) enhancement of anti-tumor immune response, which may, but is not required to, result in the regression or rejection of the tumor, or (7) relief, to some extent, of one or more symptoms associated with the cancer or tumor. The therapeutically effective amount may vary according to factors such as the disease state, age, sex and weight of the individual and the ability of one or more anti-cancer agents to elicit a desired response in the individual. A “therapeutically effective amount” is also one in which any toxic or detrimental effects are outweighed by the therapeutically beneficial effects.

The phrases “treating cancer” and “treatment of cancer” mean to decrease, reduce, or inhibit the replication of cancer cells; decrease, reduce or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; or ameliorate or alleviate the symptoms of the disease caused by the cancer.

Certain embodiments are directed toward a compound of general Formula I or a pharmaceutically acceptable salt thereof:

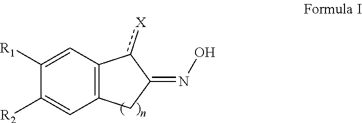

wherein, R₁ and R₂ are independently hydrogen, alkyl, branched alkyl, substituted alkyl, aryl, —CH₂—Ar, wherein Ar is aryl, substituted aryl, fused aryl, heterocyclic, or heteroaryl, optionally substituted heterocyclic or substituted heteroaryl, wherein said aryl, heterocyclic, or heteroaryl groups can be optionally substituted by one or more R₃ groups. Unless otherwise indicated, as used herein, the term “aryl” includes an organic radical derived from an aromatic hydrocarbon by removal of one hydrogen, such as phenyl (Ph), naphthyl, indenyl, indanyl, or fluorenyl. “Aryl” encompasses fused ring groups wherein at least one ring is aromatic. Unless otherwise indicated, as used herein, “heteroaryl” refers to aromatic groups containing one or more heteroatoms, preferably from one to three heteroatoms, selected from O, S, or N. A multicyclic group containing one or more heteroatoms wherein at least one ring of the group is aromatic is a “heteroaryl” group. R₃ is selected from halogen (F, Cl, Br, or I), —OH, —O—R₁, —NO₂, CO₂H, —CN, —CF₃, CO₂—R₁, CONH₂, CONHR₁, CON(R₁)₂, —NH₂, —NHR₁, —N(R₁)₂, —NHC(O)R₁, alkyl, branched alkyl, substituted alkyl, halogenated methyl (—CF₃, —CHF₂ and the like), and halogenated O-alkyl (e.g., —OCF₃). X is selected from O, —OH, N—OH, H₂, —O—R₁, bonded by a single bond or double bond as depicted. n is 1 or 2.

Certain embodiments are directed toward a compound of general Formula II or a pharmaceutically acceptable salt thereof:

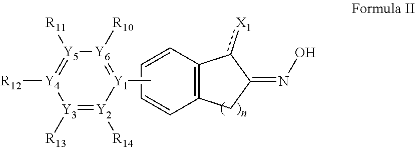

R₁₀ R₁₁, R₁₂, R₁₃, and R₁₄ are each independently selected from hydrogen; halogen (F, Cl, Br or I); hydroxyl; alkoxy; alkyl; mono-, bi-, or tri-haloalkyl; branched alkyl; nitrile; substituted alkyl, aryl (Ar), substituted Ar, —CH₂—Ar, and —CH₂-substituted AR. The aryl can be a fused aryl, heterocyclic, or heteroaryl, optionally substituted with one or more R₃ groups. R₃ is selected from halogen (F, Cl, Br and I), —OH, —O—R₄, —NO₂, CO₂H, —CN, —CF₃, CO₂—R₄, CONH₂, CONHR₄, CON(R₄)₂, —NH₂, —NHR₄, —N(R₄)₂, —NHC(O)R₄, alkyl, branched alkyl, substituted alkyl, halogenated methyl (—CF₃, —CHF₂ and the like), and halogenated O-alkyl (i.e. —OCF₃). R₄ is selected from branched or unbranched alkyl, halogenated alkyl, or heteroaryl. Unless otherwise indicated, as used herein, the term “aryl” includes an organic radical derived from an aromatic hydrocarbon by removal of one hydrogen, such as phenyl (Ph), naphthyl, indenyl, indanyl and fluorenyl. “Aryl” encompasses fused ring groups wherein at least one ring is aromatic. Unless otherwise indicated, as used herein, “heteroaryl” refers to aromatic groups containing one or more heteroatoms, preferably from one to three heteroatoms, selected from O, S and N. A multicyclic group containing one or more heteroatoms wherein at least one ring of the group is aromatic is a “heteroaryl” group. Xi is selected from O, —OH, N—OH, H₂, and —O—R₅, bonded by a single bond or double bond as depicted. Y₁, Y₂, Y₃, Y₄, Y₅, and Y₆ are independently selected from CH or N. R₅ is selected from branched or unbranched alkyl, halogenated alkyl, heteroaryl, —C(O)—R₄, and —NH—C(O)—R₄, wherein R₄ is as above, selected from branched or unbranched alkyl, halogenated alkyl, or heteroaryl. And n is 1 or 2.

The compounds of Formula I or Formula II may be prepared by the methods described below, together with synthetic methods known in the art of organic chemistry, or modifications and derivatizations that are familiar to those of ordinary skill in the art.

Preferred methods include, but are not limited to, those described below. During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Compounds of Formula I, Formula II or their pharmaceutically acceptable salts, can be prepared according to the reaction Scheme 1 below. Isolation and purification of the products is accomplished by standard procedures, which are known to a chemist of ordinary skill.

The following schemes and examples are representative of the processes for making compounds of general Formula I, wherein R₁ and/or R₂ are independently selected from H, aryl or heteroaryl; R₃ is defined as above; n is 1 or 2; X is O, —OH, N—OH or H₂. It is to be understood, however, that the invention, as fully described herein and as recited in the claims, is not intended to be limited by the details of the following examples.

Referring to Scheme 1 (Route A), condensation of bromide 1 with NH₂OH in the presence of KOAc/MeOH produces the desired oxime 2 as a mixture of geometric isomers. The bromo-oxime 2 was coupled under Suzuki conditions with the optionally substituted boronic acid 3, utilizing a silica-bound palladium catalyst (DPP-Pd), in the presence of a suitable base (K₂CO₃) in solvents such as DME/water, at temperatures ranging from room temperature to 130° C., preferably at reflux, which provides the biaryl oxime 4.

The synthesis of the corresponding 6-aryl-keto-oximes 7 and 6-aryl-hydroxyl-oximes 8 are highlighted in Route B in Scheme 1. Reaction of bromo-ketone 5 with isopentyl nitrite in the presence of HCl produces the corresponding keto-oxime 6. The bromo-oxime 6 was coupled under Suzuki conditions with the optionally substituted boronic acid 3, utilizing Pd(OAc)₂ and RuPhos catalyst in the presence of K₂PO₄ in dioxane/water, under microwave irradiation conditions, at temperatures ranging from room temperature to 130° C., preferably at reflux, which provides the desired biaryl keto-oxime 7. Reduction with NaBH₄ produces the corresponding hydroxyl-oxime derivatives 8.

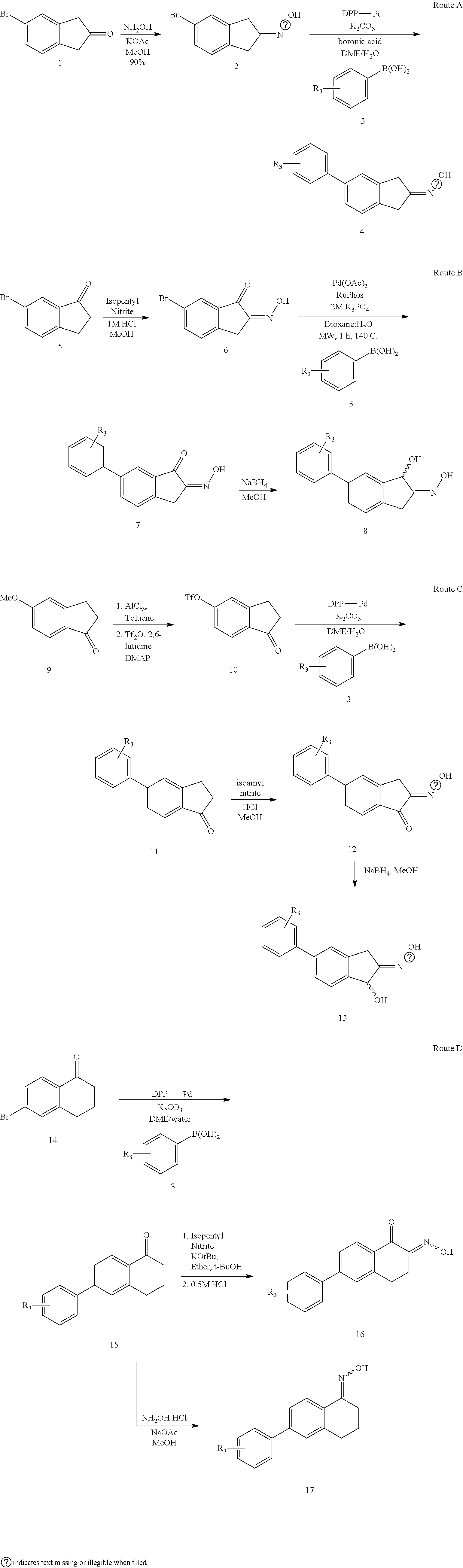

Scheme 1. Synthesis Routes A-D. Route C in Scheme 1 details the synthesis of the corresponding 5-aryl-keto-oximes 12 and 5-aryl-hydroxyl-oximes 13. Referring to Route C in Scheme 1,5-methoxyindanone 9 was treated with AlCl₃ in toluene to provide the corresponding 5-hydroxyindanone (not depicted), which was reacted with Tf₂O in the presence of a pyridine base such as 2,6-lutidiene and 4-DMAP to provide the desired triflate 10. The triflate 10 was coupled under Suzuki conditions with the optionally substituted boronic acid 3, utilizing a silica-bound palladium catalyst (DPP-Pd), in the presence of a suitable base (K₂CO₃) in solvents such as DME/water, at temperatures ranging from room temperature to 130° C., preferably at reflux, which provides the biaryl ketone 11. Reaction of ketone 11 with isoamyl nitrite in the presence of HCl/MeOH provides the desired 5-aryl-keto-oxime 12. Reduction of 12 with NaBH₄ produces the corresponding 5-aryl-hydroxyl-oxime derivatives 13.

Route D in Scheme 1 details the synthesis of the corresponding tetralone derivatives 16 and 17. Referring to Route D, the bromo-tetralone 14 was coupled under Suzuki conditions with the optionally substituted boronic acid 3, utilizing a silica-bound palladium catalyst (DPP-Pd), in the presence of a suitable base (K₂CO₃) in solvents such as DME/water, at temperatures ranging from room temperature to 130° C., preferably at reflux, which provides the biaryl tetralone 15. Reaction of ketone 15 with isopentyl nitrite in the presence of KO-tBu and t-BuOH, followed by HCl/MeOH provides the desired tetralone keto-oxime derivatives 16. Alternatively, 15 can be condensed with NH₂OH to provide the tetralone oxime 17.

Pharmaceutically acceptable salts of the compounds of Formula I include the acid or base addition salts thereof. All three reactions are typically carried out in solution. The resulting salt may precipitate out and be collected by filtration or may be recovered by evaporation of the solvent. The degree of ionization in the resulting salt may vary from completely ionized to almost non-ionized. Suitable non-toxic, acid-addition pharmaceutically acceptable salts include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable non-toxic, base-addition pharmaceutically acceptable salts include, but are not limited to, aluminium, arginine, benzathine, calcium, choline, diethylamine, diolamine, glycine, lysine, magnesium, meglumine, olamine, potassium, sodium, tromethamine and zinc salts. For a review on suitable salts, see Handbook of Pharmaceutical Salts: Properties, Selection, and Use by Stahl and Wermuth (Wiley-VCH, 2002).

Included within the scope of the present invention are all stereoisomers, geometric isomers and tautomeric forms of the compounds of Formula I, including compounds exhibiting more than one type of isomerism, and mixtures of one or more thereof.

The present invention includes all pharmaceutically acceptable isotopically-labeled compounds of Formula I wherein one or more atoms are replaced by atoms having the same atomic number, but an atomic mass or mass number different from the atomic mass or mass number which predominates in nature.

Chemical Definitions—Various chemical definitions related to such compounds are provided as follows.

As used herein, “predominantly one enantiomer” means that the compound contains at least 85% of one enantiomer, or more preferably at least 90% of one enantiomer, or even more preferably at least 95% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer.

As used herein, the term “water soluble” means that the compound dissolves in water at least to the extent of 0.010 mole/liter or is classified as soluble according to literature precedence.

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxyl” means —OH.

The term “alkyl,” by itself or as part of another substituent, means, unless otherwise stated, a linear (i.e., unbranched) or branched carbon chain, which may be fully saturated, monounsaturated or polyunsaturated. An unsaturated alkyl group is one having one or more double bonds or triple bonds. Unsaturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Examples of optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g., “C₁₋₄ alkylsulfonyl”)

The term “monosaccharide” refers to a cyclized monomer unit based on a compound having a chemical structure H(CHOH)ₙC(═O)(CHOH)ₘH wherein n+m is 4 or 5. Thus, monosaccharides include, but are not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose.

An “isomer” of a first compound is a separate compound in which each molecule contains the same constituent atoms as the first compound, but where the configuration of those atoms in three dimensions differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out). All compounds with an asterisk (*) adjacent to a tertiary or quarternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of Formula I or Formula II includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine 19 is longest-lived. Thus, an isotope of a compound having the structure of Formula (I) includes, but not limited to, compounds of Formula (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

It is contemplated that any embodiment discussed in this specification can be implemented with respect to any method or composition of the invention, and vice versa. Furthermore, compositions of the invention can be used to achieve methods of the invention.

### I. Methods of Treating Cancer

Certain embodiments are directed to methods of treating cancer by administering one or more compounds described above. In the methods for treating cancer provided herein, the compounds described above are administered in effective amounts. An effective amount means that amount necessary to delay the onset of, inhibit the progression, halt altogether the onset or progression, or diagnose the particular cancer being treated. When administered to a subject, effective amounts will depend on the particular condition being treated, the severity of the condition, individual patient parameters including age, physical condition, size and weight, concurrent treatment, frequency of treatment, and the mode of administration. These factors are well known to those of ordinary skill in the art and can be addressed with no more than routine experimentation. It is preferred generally that a maximum dose be used, that is, the highest safe dose according to some medical judgment.

In certain embodiments, the invention also provides compositions comprising 1, 2, 3 or more anti-cancer agents with one or more of the following: a pharmaceutically acceptable diluent; a carrier; a solubilizer; an emulsifier; a preservative; and/or an adjuvant. Such compositions may contain an effective amount of at least one anti-cancer agent. Thus, the use of one or more anti-cancer agents that are provided herein in the preparation of a pharmaceutical composition of a medicament is also included. Such compositions can be used in the treatment of a variety of cancers. In certain embodiments the treatment is for brain, breast, ovarian, prostate, or salivary cancer.

The anti-cancer agents may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the anti-cancer agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The compositions described herein can be administered using conventional modes of delivery including, but not limited to intravenous, intraperitoneal, oral, subcutaneous administration, intraarterial, intramuscular, intrapleural, intrathecal, and by perfusion through a regional catheter. Local administration to a tumor in question is also contemplated by the present invention. When administering the compositions by injection, the administration may be by continuous infusion or by single or multiple boluses. For parenteral administration, the anti-cancer agents may be administered in a pyrogen-free, parenterally acceptable aqueous solution comprising the desired anti-cancer agents in a pharmaceutically acceptable vehicle. A particularly suitable vehicle for parenteral injection is sterile distilled water in which one or more anti-cancer agents are formulated as a sterile, isotonic solution, properly preserved.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental administration are also sterile, substantially isotonic and made under GMP conditions.

For the compounds of the present invention, alone or as part of a pharmaceutical composition, such doses are between about 0.001, 0.01, 0.1, 0.5 mg/kg and 0.6, 0.7, 0.8, 0.9, 1 mg/kg body weight. In certain aspects, a dose is between about 1 and 100 mg/kg body weight, most preferably between 1 and 10 mg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

In some methods of the invention, the cancer cell is a tumor cell. The cancer cell may be in a patient. The patient may have a solid tumor. In such cases, embodiments may further involve performing surgery on the patient, such as by resecting all or part of the tumor. Compositions may be administered to the patient before, after, or at the same time as surgery. In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Methods of treating cancer may further include administering to the patient chemotherapy or radiotherapy, which may be administered more than one time. Chemotherapy includes, but is not limited to, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, topotecan, or any analog or derivative variant thereof. Radiation therapy includes, but is not limited to, X-ray irradiation, UV-irradiation, γ-irradiation, electron-beam radiation, or microwaves. Moreover, a cell or a patient may be administered a microtubule stabilizing agent, including, but not limited to, taxane, as part of methods of the invention. It is specifically contemplated that any of the compounds or derivatives or analogs, can be used with these combination therapies.

In some embodiments, the cancer that is administered the composition(s) described herein may be a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterus cell.

### II. Methods of Neuroprotection

ERβ levels can dictate both synaptic strength and neuroplasticity through neural structure modifications. Variations in endogenous estrogen levels cause changes in dendritic architecture in the hippocampus, which affects neural signaling and plasticity. Specifically, lower estrogen levels lead to decreased dendritic spines and improper signaling, inhibiting plasticity of the brain. However, treatment with a ERβ agonist can reverse this affect. As a result of the relationship between dendritic architecture and long-term potentiation (LTP), ERβ can enhance LTP and lead to an increase in synaptic strength. Furthermore, ERβ agonist can promote neurogenesis in developing hippocampal neurons and neurons in the subventricular zone and dentate gyrus of the adult human brain. Specifically, ERβ increases the proliferation of progenitor cells to create new neurons and can be increased later in life through ERβ agonist treatment.

### III. EXAMPLES

The following examples as well as any figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I

### Specificity of the New ERβ Agonists to ERα Vs ERβ Determined Using ERE Reporter Assays

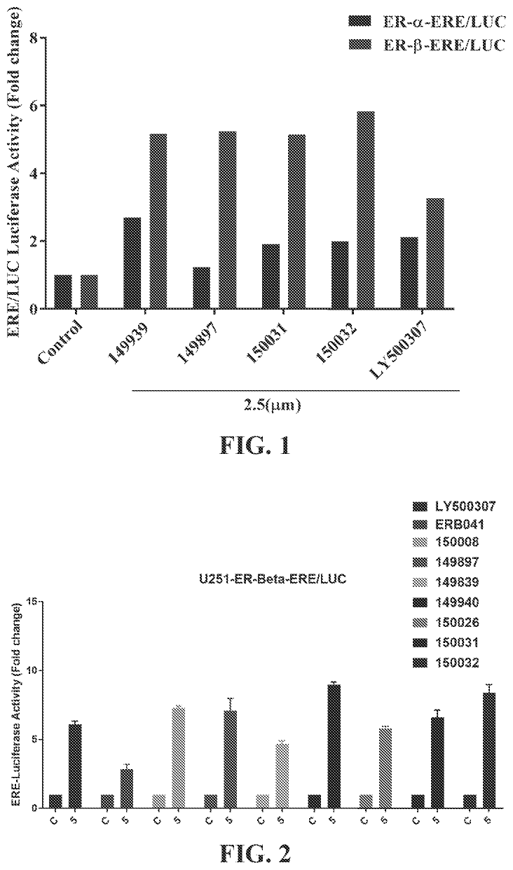

To examine the specificity of new ERβ agonist towards ERα and ERβ, a HEK 293 ERE reporter assay was used. HEK293 cells do not express ERα or ERβ. Cells were transiently transfected with ERα or ERβ along with ERE reporter. ERE reporter contains estrogen response element (ERE) driven luciferase gene. When ERα or ERβ is bound by an agonist, they translocate to the nucleus, bind ERE element and contribute to transcription of the luciferase gene. Addition of luciferin to these cells results in bioluminescence. Candidate compounds were added to these cells and ERE-dependent luciferase activity was measured. Published ERβ agonist LY5000307 (CAS #533884-09-2; (3aS,4R,9bR)-4-(4-hydroxyphenyl)-1,2,3,3a,4,9b-hexahydrocyclopenta[c]chromen-8-ol) was used as a positive control. ERβ agonists described herein showed higher magnitude of activation of ERβ compared to ERα and showed better potency in activation of ERβ compared to existing ERβ agonist LY5000307 (LY) (FIG. 1).

### Example 2

### Specificity of the ERβ Agonist in Glioblastoma Multiform (GBM, Brain Tumor) Model Cells.

ERβ specific activity was confirmed utilizing glioblastoma U251 cancer cells that stably express estrogen response element (ERE) driven luciferase gene. These cells do not express the estrogen receptor alpha (ERα), however, uniquely express ERβ. When ERβ is bound by an agonist and activated, it translocates to the nucleus where it interacts with the ERE, leading to transcription of the luciferase gene. Addition of luciferin to these cells results in bioluminescence. Candidate compounds were added at various concentrations and revealed ERE-dependent luciferase activity at 5 μM (FIG. 2). Previously published ERβ agonists (LY5000307; ERB041 (CAS #524684-52-4, 2-(3-fluoro-4-hydroxyphenyl)-7-vinylbenzo[d]oxazol-5-ol)), were included as positive controls. These results showed that new candidate compounds function as potent ERβ agonists in GBM model cells.

### Example 3

### Biological Activity of New Compounds in GBM Cells.

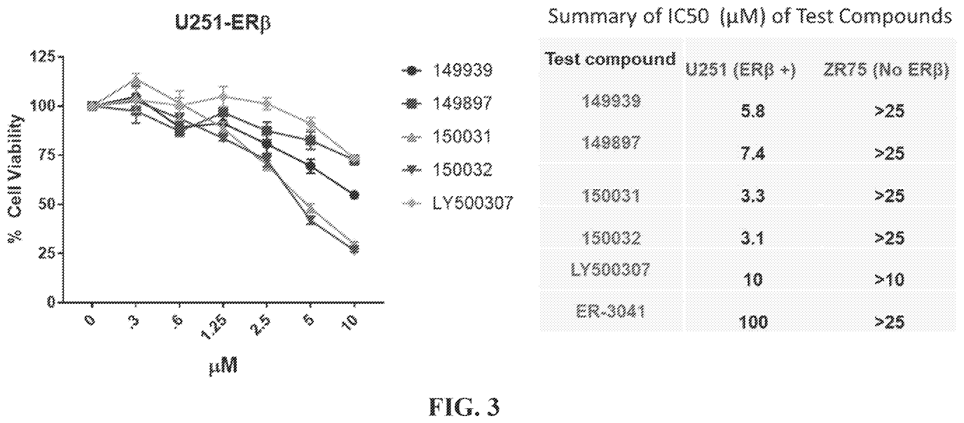

To assess, the antitumor activity of the new ERβ agonist compounds, GBM cells were assessed for viability using a commercially available MTT assay. Briefly, the assay determines the number of viable cells in culture by quantifying ATP, which indicates the presence of metabolically active cells. Previously described agonists such as LY was able to reduce viability by 50% with concentration of 10 μM or greater. The new compounds described in this disclosure have higher potency than existing compounds and some reduce cell viability by 50% at concentrations even at 3 μM (FIG. 3). Further, the new ERβ agonists did not showed any activity in ERα expressing ZR75 cells, confirming that these new compounds lack ERα activity, thus likely will have lesser side effects through ERα (FIG. 3 right panel).

### Example 4

### Specificity of New Compounds to ERβ Versus Era Determined Using Biophysical Assay

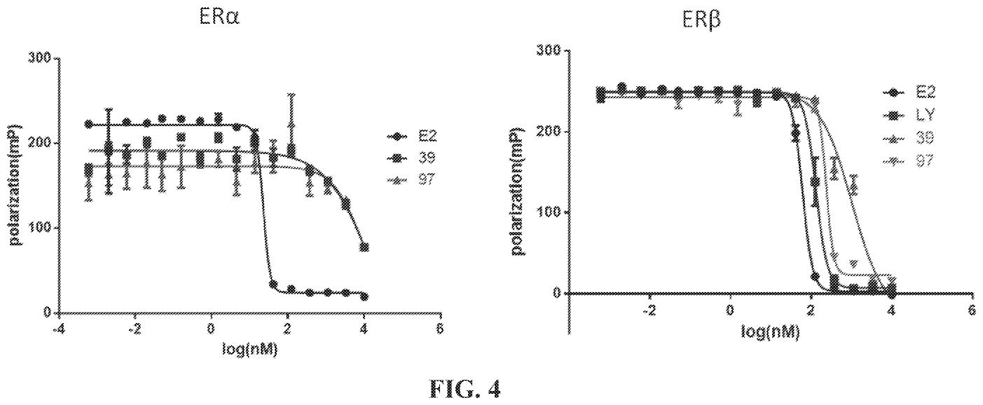

To assay specificity of ERβ versus ERα, a commercially available Polar Screen Nuclear Receptor (NR) Competitive Binding Assay (Thermo Fisher Scientific) was used. When the NR binds to the Fluormone ligand, the resulting complex yields a high polarization value. If the test compound displaces the Fluormone ligand from the complex, the polarization value is lowered. Since this occurs only in the presence of a test compound, the shift in polarization value enables one to accurately and conveniently determine relative affinity of a test compound for the NR. Separate assays are available for the ERα and ERβ. Results showed higher selectivity of new ERβ agonists to ERβ by 29 to 40 fold compared to ERα (FIG. 4)

### Example 5

### Specificity of the New ERβ Agonists to ERβ was Confirmed Using ERβ Knockout Cells

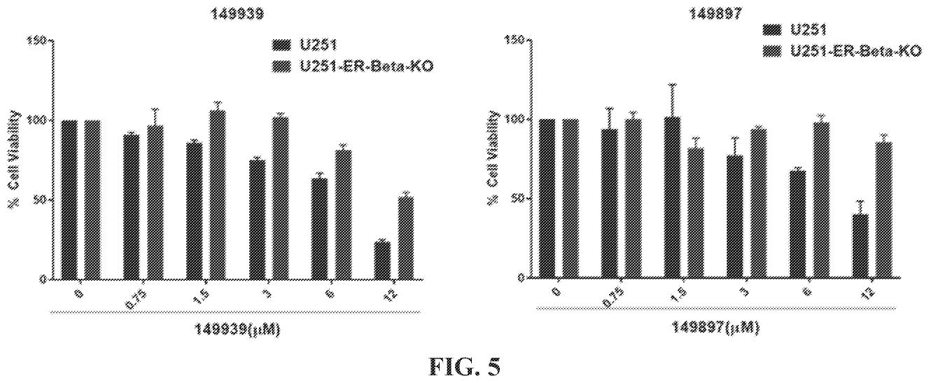

The specificity of ERβ agonists acting in cell viability assays was tested using GBM cells in which ERβ was knocked out using CRISPR/Cas9 system. The ability of ERβ agonists to reduce cell viability was significantly reduced when ERβ is knocked out (FIG. 5). These results suggest that the activity seen by the new ERβ agonists is indeed depended on ERβ.

### Example 6

Activity of the Test Compounds to Ovarian Cancer (OC_{A}) Cells that Express ERβ

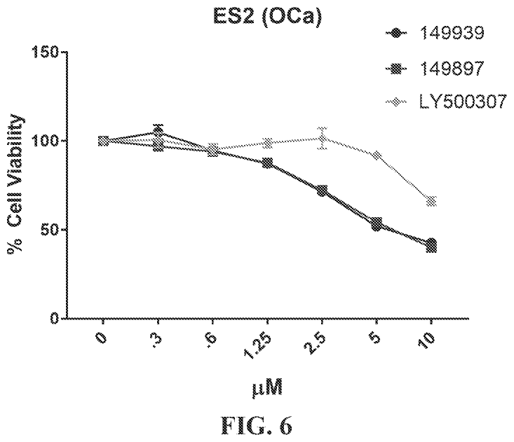

Earlier studies showed that OCa express ERβ and agonists ERβ significantly reduces viability of OCa cells. In this assay, the ability of new ERβ agonists to reduce the growth of OCa cells was tested. Results showed that new ERβ agonist are more potent than the existing ERβ agonist LY in reducing the cell viability of OCa cells (FIG. 6).

### Example 7

### Synthetic Examples

The invention is illustrated in the following non-limiting examples.

General procedures. All operations were carried out at room or ambient temperature, that is, in the range of 18-25° C.; evaporation of solvent was carried out using a rotary evaporator under reduced pressure with a bath of up to 50° C.; reactions were monitored by thin layer chromatography (tlc) and reaction times are given for illustration only. Unless otherwise indicated all reactions were conducted in standard commercially available glassware using standard synthetic chemistry methods and setup. All air- and moisture-sensitive reactions were performed under nitrogen atmosphere with dried solvents and glassware under anhydrous conditions. Starting materials and reagents were commercial compounds of the highest purity available and were used without purification (See list of specific reagents below). Solvents used for reactions were indicated as of commercial dry or extra-dry or analytical grade. Analytical thin layer chromatography was performed on aluminium plates coated with Merck Kieselgel 60F254 and visualized by UV irradiation (254 nm) or by staining with a solution of potassium permanganate. Flash column chromatography was performed on Biotage Isolera One 2.2 using commercial columns that were pre-packed with Merck Kieselgel 60 (230-400 mesh) silica gel. Final compounds for biological testing are all ≥95% purity as determined by HPLC-MS and ¹H NMR. ¹H NMR experiments were recorded on Agilent DD2 400 MHz spectrometers at ambient temperature. Samples were dissolved and prepared in deuterated solvents (CDCl₃, CD₃OD and DMSOd₆) with residual solvents being used as the internal standard in all cases. All deuterated solvent peaks were corrected to the standard chemical shifts (CDCl₃, dH=7.26 ppm; CD₃OD, dH=3.31 ppm; DMSOd₆, dH=2.50 ppm). Spectra were all manually integrated after automatic baseline correction. Chemical shifts (d) are given in parts per million (ppm) and coupling constants (J) are given in Hertz (Hz). The proton spectra are reported as follows: d (multiplicity, coupling constant J, number of protons). The following abbreviations were used to explain the multiplicities: app=apparent, b=broad, d=doublet, dd=doublet of doublets, ddd=doublet of doublet of doublets, dddd=doublet of doublet of doublet of doublets, m=multiplet, s=singlet, t=triplet. All samples were analyzed on Agilent 1290 series HPLC system comprised of binary pumps, degasser and UV detector, equipped with an auto-sampler that is coupled with Agilent 6150 mass spectrometer. Purity was determined via UV detection with a bandwidth of 170 nm in the range from 230-400 nm. The general LC parameters were as follows: Column—Zorbax Eclipse Plus C18, size 2.1×50 mm; Solvent A: 0.10% formic acid in water, Solvent B: 0.00% formic acid in acetonitrile; Flow rate—0.7 mL/min; Gradient: 5% B to 95% B in 5 min and hold at 95% B for 2 min; UV detector—channel 1=254 nm, channel 2=254 nm. Mass detector Agilent Jet Stream—Electron Ionization (AJS-ES).

The following abbreviations are used: THF—tetrahydrofuran, DCM or CH₂Cl₂-dichloromethane, DCE—dichloroethane, NaHCO₃— sodium bicarbonate, HCl—hydrogen chloride, MgSO₄—magnesium sulfate, Na₂SO₄— sodium sulfate, DME—dimethoxyethane, n-BuLi—n-butyllithium, DMF—dimethylformamide, DMSO—dimethylsulfoxide, Et₂O—diethyl ether, MeOH—methanol, EtOAc—ethyl acetate.

The IUPAC names, structures, CIDD #'s and lot #'s for individual compounds and specific examples screened for ERβ inhibition and in vitro activity are listed in the table below.

| | | | MW | Observed | |
|---|---|---|---|---|---|
| Molecule Name | Structure | IUPAC | (g/mol) | m/z | Lot ID |
| CIDD-0149897 | 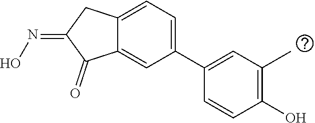 [SMILES: Cc1cc(-c2ccc3c(c2)C(=O)/C(=N\O)C3)ccc1O] | (2Z)-6-(3-fluoro-4- hydroxyphenyl)-2- (hydroxyimino)- 2,3-dihydro-1H- inden-1-one | 271.247 | 270.0 | SM2019-99-77 |
| CIDD-0149898 | 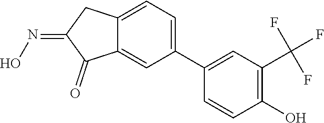 [SMILES: O=C1/C(=N\O)Cc2ccc(-c3ccc(O)c(C(F)(F)F)c3)cc21] | (2Z)-6-[4-hydroxy- 3-(trifluoro- methyl)phenyl]- 2-(hydroxyimino)- 2,3-dihydro-1H- inden-1-one | 321.255 | 319.8 | SM2017-74-91 |
| CIDD-0149899 | 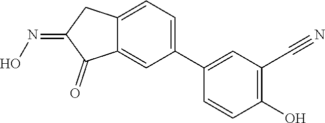 [SMILES: N#Cc1cc(-c2ccc3c(c2)C(=O)/C(=N\O)C3)ccc1O] | 2-hydroxy-5-[(2Z)-2- (hydroxyimino)-3- oxo-2,3-dihydro-1H- inden-5- yl]benzonitrile | 278.267 | 276.9 | SM2017-74-60 |
| CIDD-0149900 | 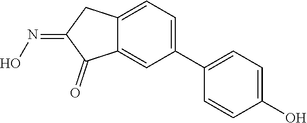 [SMILES: O=C1/C(=N\O)Cc2ccc(-c3ccc(O)cc3)cc21] | (2Z)-2- (hydroxyimino)-6- (4-hydroxyphenyl)- 2,3-dihydro-1H- inden-1-one | 253.257 | 254.1 | SM2017-74-67 |
| CIDD-0149900 | 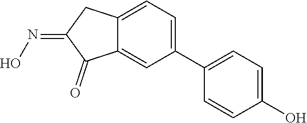 [SMILES: O=C1/C(=N\O)Cc2ccc(-c3ccc(O)cc3)cc21] | (2Z)-2- (hydroxyimino)-6- (4-hydroxyphenyl)- 2,3-dihydro-1H- inden-1-one | 253.257 | 254.2 | SM2017-74-107 |
| CIDD-0149933 | 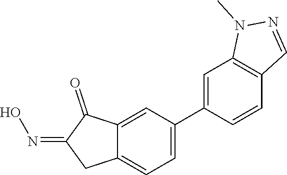 [SMILES: Cn1ncc2ccc(-c3ccc4c(c3)C(=O)/C(=N\O)C4)cc21] | (2Z)-2- (hydroxyimino)-6- (1-methyl-1H- indazol-6-yl)-2,3- dihydro-1H-inden- 1-one | 291.31 | 292.2 | SM2017-74-105 |
| CIDD-0149934 | 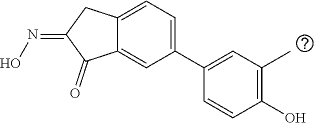 [SMILES: Cc1cc(-c2ccc3c(c2)C(=O)/C(=N\O)C3)ccc1O] | (2Z)-6-(3-chloro- 4-hydroxyphenyl)- 2-(hydroxyimino)- 2,3-dihydro-1H- inden-1-one | 287.7 | 288.1 | SM2017-74-58 |
| CIDD-0149934 | 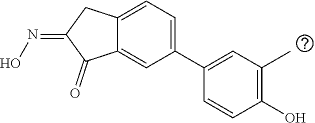 [SMILES: Cc1cc(-c2ccc3c(c2)C(=O)/C(=N\O)C3)ccc1O] | (2Z)-6-(3-chloro- 4-hydroxyphenyl)- 2-(hydroxyimino)- 2,3-dihydro-1H- inden-1-one | 287.7 | 288.1 | SM2017-74-87 |
| CIDD-0149934 | 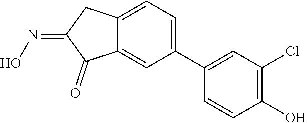 [SMILES: O=C1/C(=N\O)Cc2ccc(-c3ccc(O)c(Cl)c3)cc21] | (2Z)-6-(3-chloro- 4-hydroxyphenyl)- 2-(hydroxyimino)- 2,3-dihydro-1H- inden-1-one | 287.7 | 289.2 | SM2017-74-66 |
| CIDD-0149936 | 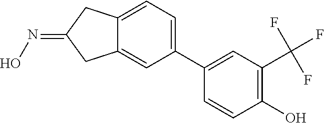 [SMILES: O/N=C1/Cc2ccc(-c3ccc(O)c(C(F)(F)F)c3)cc2C1] | 4-[(2Z)-2- (hydroxyimino)- 2,3-dihydro-1H- inden-5-yl]-2- (trifluorometh- yl)phenol | 307.272 | 349.2 (+CH3CN) | SM2017-74-90 |
| CIDD-0149938 | 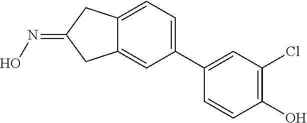 [SMILES: O/N=C1/Cc2ccc(-c3ccc(O)c(Cl)c3)cc2C1] | 2-chloro-4-[(2Z)- 2-(hydroxyimino)- 2,3-dihydro-1H- inden-5-yl]phenol | 273.72 | 315.1 (+CH3CN) | SM2017-74-101 |
| CIDD-0149940 | 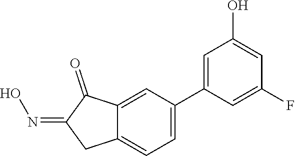 [SMILES: O=C1/C(=N\O)Cc2ccc(-c3cc(O)cc(F)c3)cc21] | (2Z)-6-(3-fluoro- 5-hydroxyphenyl)- 2-(hydroxyimino)- 2,3-dihydro-1H- inden-1-one | 271.247 | 272.1 | SM2017-74-104 |
| CIDD-0149969 | 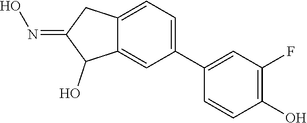 [SMILES: O/N=C1\Cc2ccc(-c3ccc(O)c(F)c3)cc2C1O] | (2E)-6-(3-fluoro-4- hydroxyphenyl)-2- (hydroxyimino)- 2,3-dihydro-1H- inden-1-ol | 273.263 | 256.2 | SM2017-74-123 |
| CIDD-0149970 | 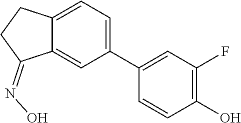 [SMILES: O/N=C1/CCc2ccc(-c3ccc(O)c(F)c3)cc21] | 2-fluoro-4-[3- (hydroxyimino)- 2,3-dihydro-1H- inden-5-yl]phenol | 257.264 | 258.2 | SM2017-74-125 |
| CIDD-0149971 | 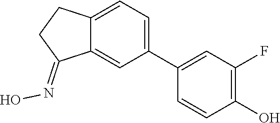 [SMILES: O/N=C1\CCc2ccc(-c3ccc(O)c(F)c3)cc21] | 2-fluoro-4-[(3E)- 3-(hydroxyimino)- 2,3-dihydro-1H- inden-5-yl]phenol | 257.264 | 299.2 | SM2017-74-122 |
| CIDD-0149972 | 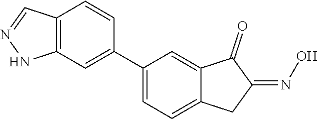 [SMILES: O=C1/C(=N\O)Cc2ccc(-c3ccc4cn[nH]c4c3)cc21] | (2Z)-2- (hydroxyimino)-6- (1H-indazol-6-yl)- 2,3-dihydro-1H- inden-1-one | 277.283 | 319.2 (+CH3CN) | SM2017-74-137 |
| CIDD-0149998 | 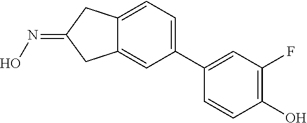 [SMILES: O/N=C1/Cc2ccc(-c3ccc(O)c(F)c3)cc2C1] | 2-fluoro-4-[(2Z)- 2-(hydroxyimino)- 2,3-dihydro-1H- inden-5-yl]phenol | 257.264 | 299.2 (+CH3CN) | SM2017-74-121 |
| CIDD-0149999 | 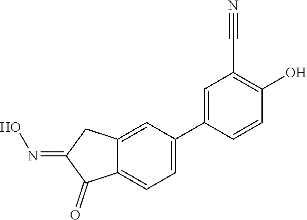 [SMILES: N#Cc1cc(-c2ccc3c(c2)C/C(=N\O)C3=O)ccc1O] | 2-hydroxy-5- [(2E)-2- (hydroxyimino)-1- oxo-2,3-dihydro- 1H-inden-5- yl]benzonitrile | 278.267 | 276.9 | SM2017-74-129 |
| CIDD-0150000 | 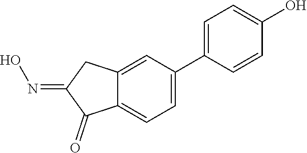 [SMILES: O=C1/C(=N/O)Cc2cc(-c3ccc(O)cc3)ccc21] | (2E)-2- (hydroxyimino)-5- (4- hydroxyphenyl)- 2,3-dihydro- 1H-inden-1-one | 253.257 | 254.2 | SM2017-74-130 |
| CIDD-0150001 | 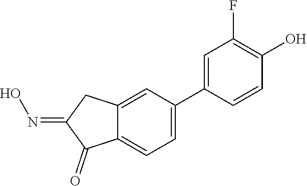 [SMILES: O=C1/C(=N/O)Cc2cc(-c3ccc(O)c(F)c3)ccc21] | (2E)-5-(3-fluoro- 4-hydroxyphenyl)- 2-(hydroxyimino)- 2,3-dihydro-1H- inden-1-one | 271.247 | 269.9 | SM2017-74-131 |
| CIDD-0150002 | 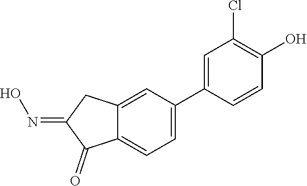 [SMILES: O=C1/C(=N/O)Cc2cc(-c3ccc(O)c(Cl)c3)ccc21] | (2E)-5-(3-chloro- 4-hydroxyphenyl)- 2-(hydroxyimino)- 2,3-dihydro-1H- inden-1-one | 287.7 | 285.8 | SM2017-74-132 |
| CIDD-0150003 | 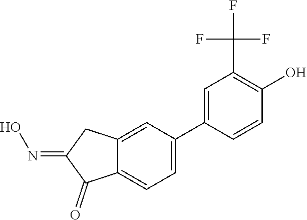 [SMILES: O=C1/C(=N/O)Cc2cc(-c3ccc(O)c(C(F)(F)F)c3)ccc21] | (2E)-5-[4- hydroxy-3- (trifluorometh- yl)phenyl]-2- (hydroxyimino)- 2,3-dihydro-1H- inden-1-one | 321.255 | 319.8 | SM2017-74-133 |
| CIDD-0150004 | 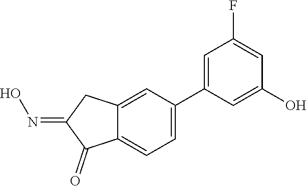 [SMILES: O=C1/C(=N/O)Cc2cc(-c3cc(O)cc(F)c3)ccc21] | (2E)-5-(3-fluoro- 5-hydroxyphenyl)- 2-(hydroxyimino)- 2,3-dihydro-1H- inden-1-one | 271.247 | 315.9 (+CH3CN) | SM2017-74-134 |
| CIDD-0150005 | 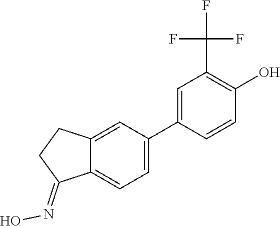 [SMILES: O/N=C1\CCc2cc(-c3ccc(O)c(C(F)(F)F)c3)ccc21] | 4-[(1E)-1- (hydroxyimino)- 2,3-dihydro-1H- inden-5-yl]-2- (trifluorometh- yl)phenol | 307.272 | 308.2 | SM2017-74-138 |
| CIDD-0150006 | 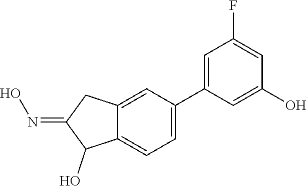 [SMILES: O/N=C1\Cc2cc(-c3cc(O)cc(F)c3)ccc2C1O] | (2E)-5-(3-fluoro- 5-hydroxyphenyl)- 2-(hydroxyimino)- 2,3-dihydro-1H- inden-1-ol | 273.263 | 256.2 | SM2017-74-139 |
| CIDD-0150007 | 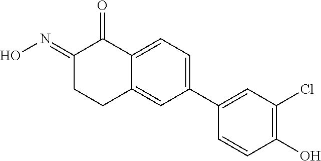 [SMILES: O=C1/C(=N/O)CCc2cc(-c3ccc(O)c(Cl)c3)ccc21] | (2E)-6-(3-chloro- 4-hydroxyphenyl)- 2-(hydroxyimino)- 1,2,3,4-tetrahydro- naphthalen-1-one | 301.73 | 302.1 | SM2017-74-142 |
| CIDD-0150008 | 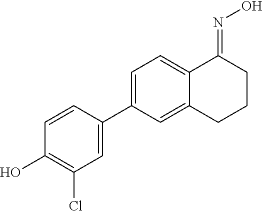 [SMILES: O/N=C1\CCCc2cc(-c3ccc(O)c(Cl)c3)ccc21] | 2-chloro-4-[(SE)- 5-(hydroxyimino)- 5,6,7,8-tetrahydro- naphthalen-2- yl]phenol | 287.74 | 288.2 | SM2017-74-143 |
| CIDD-0150018 | 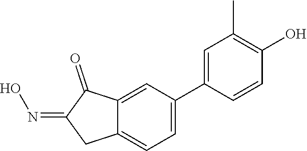 [SMILES: Cc1cc(-c2ccc3c(c2)C(=O)/C(=N\O)C3)ccc1O] | (2Z)-6-(4-hydroxy- 3-methylphenyl)-2- (hydroxyimino)- 2,3-dihydro-1H- inden-1-one | 267.284 | 268.2 | SM2017-74-148 |
| CIDD-0150019 | 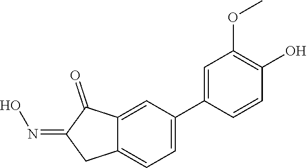 [SMILES: COc1cc(-c2ccc3c(c2)C(=O)/C(=N\O)C3)ccc1O] | (2Z)-6-(4-hydroxy- 3-methoxyphenyl)- 2-(hydroxyimino)- 2,3-dihydro-1H- inden-1-one | 283.283 | 284.2 | SM2017-74-149 |
| CIDD-0150020 | 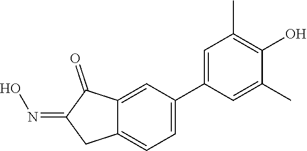 [SMILES: Cc1cc(-c2ccc3c(c2)C(=O)/C(=N\O)C3)cc(C)c1O] | (2Z)-6-(4- hydroxy-3,5- dimethylphenyl)- 2-(hydroxyimino)- 2,3-dihydro-1H- inden-1-one | 281.311 | 282.2 | SM2017-74-150 |
| CIDD-0150021 | 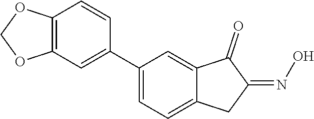 [SMILES: O=C1/C(=N\O)Cc2ccc(-c3ccc4c(c3)OCO4)cc21] | (2Z)-6-(2H-1,3- benzodioxol-5- yl)-2- (hydroxyimino)- 2,3-dihydro-1H- inden-1-one | 281.267 | 282.1 | SM2017-74-152 |
| CIDD-0150022 | 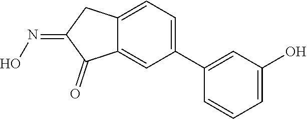 [SMILES: O=C1/C(=N\O)Cc2ccc(-c3cccc(O)c3)cc21] | (2Z)-2- (hydroxyimino)-6- (3-hydroxyphenyl)- 2,3-dihydro-1H- inden-1-one | 253.257 | 254.2 | SM2017-74-153 |
| CIDD-0150023 | 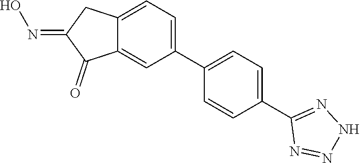 [SMILES: O=C1/C(=N/O)Cc2ccc(-c3ccc(-c4nn[nH]n4)cc3)cc21] | (2E)-2- (hydroxyimino)-6- [4-(2H-1,2,3,4- tetrazol-5- yl)phenyl]-2,3- dihydro-1H-inden- 1-one | 305.297 | 303.9 | SM2017-74-159 |
| CIDD-0150024 | 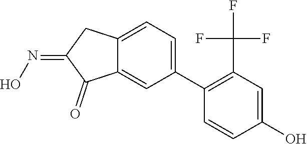 [SMILES: O=C1/C(=N\O)Cc2ccc(-c3ccc(O)cc3C(F)(F)F)cc21] | (2Z)-6-[4-hydroxy- 2-(trifluorometh- yl)phenyl]-2- (hydroxyimino)- 2,3-dihydro-1H- inden-1-one | 321.255 | 319.6 | SM2017-74-169-1 |
| CIDD-0150025 | 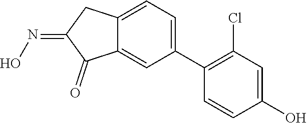 [SMILES: O=C1/C(=N\O)Cc2ccc(-c3ccc(O)cc3Cl)cc21] | (2Z)-6-(2-chloro- 4-hydroxyphenyl)- 2-(hydroxyimino)- 2,3-dihydro-1H- inden-1-one | 287.7 | 285.9 | SM2017-74-169-2 |
| CIDD-0150026 | 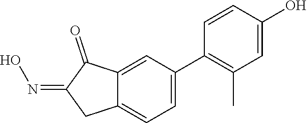 [SMILES: Cc1cc(O)ccc1-c1ccc2c(c1)C(=O)/C(=N\O)C2] | (2Z)-6-(4- hydroxy-2- methylphenyl)-2- (hydroxyimino)- 2,3-dihydro-1H- inden-1-one | 267.284 | 268.2 | SM2017-74-169-3 |
| CIDD-0150027 | 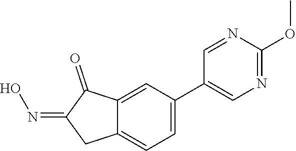 [SMILES: COc1ncc(-c2ccc3c(c2)C(=O)/C(=N\O)C3)cn1] | (2Z)-2- (hydroxyimino)-6- (2- methoxypyrimidin- 5-yl)-2,3-dihydro- 1H-inden-1-one | 269.26 | 270.2 | SM2017-74-169-4 |
| CIDD-0150028 | 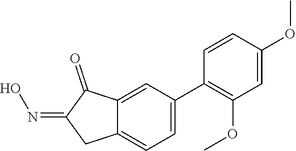 [SMILES: COc1ccc(-c2ccc3c(c2)C(=O)/C(=N\O)C3)c(OC)c1] | (2Z)-6-(2,4- dimethoxyphenyl)- 2-(hydroxyimino)- 2,3-dihydro-1H- inden-1-one | 297.31 | 298.2 | SM2017-74-169-5 |
| CIDD-0150029 | 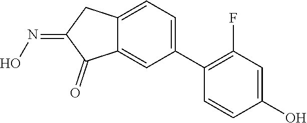 [SMILES: O=C1/C(=N\O)Cc2ccc(-c3ccc(O)cc3F)cc21] | (2Z)-6-(2-fluoro- 4-hydroxyphenyl)- 2-(hydroxyimino)- 2,3-dihydro-1H- inden-1-one | 271.247 | 269.9 | SM2017-74-169-6 |
| CIDD-0150030 | 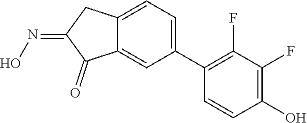 [SMILES: O=C1/C(=N\O)Cc2ccc(-c3ccc(O)c(F)c3F)cc21] | (2Z)-6-(2,3- difluoro-4- hydroxyphenyl)-2- (hydroxyimino)- 2,3-dihydro-1H- inden-1-one | 289.238 | 287.8 | SM2017-74-169-7 |
| CIDD-0150031 | 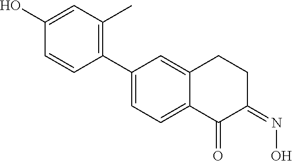 [SMILES: Cc1cc(O)ccc1-c1ccc2c(c1)CC/C(=N/O)C2=O] | (2Z)-6-(4- hydroxy-2- methylphenyl)-2- (hydroxyimino)- 1,2,3,4- tetrahydronapthal- en-1-one | 281.311 | 282.2 | SM2017-74-169-8 |
| CIDD-0150032 | 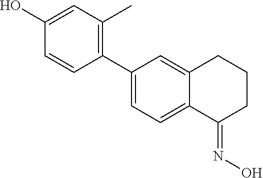 [SMILES: Cc1cc(O)ccc1-c1ccc2c(c1)CCC/C2=N\O] | 4-[(5E)-5- (hydroxyimino)- 5,6,7,8- tetrahydronapthal- en-2-yl]-3- methylphenol | 267.328 | 268.2 | SM2017-74-172 |
| CIDD-0150033 | 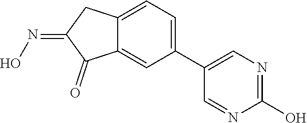 [SMILES: O=C1/C(=N\O)Cc2ccc(-c3cnc(O)nc3)cc21] | (2Z)-2- (hydroxyimino)-6- (2- hydroxypyrimidin- 5-yl)-2,3-dihydro- 1H-inden-1-one | 255.233 | 256.2 | SM2017-74-170 |
| CIDD-0150168 | 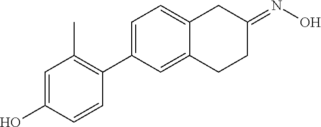 [SMILES: Cc1cc(O)ccc1-c1ccc2c(c1)CC/C(=N\O)C2] | 4-[(6E)-6- (hydroxyimino)- 5,6,7,8- tetrahydronapthal- en-2-yl]-3- methylphenol | 267.328 | 268.2 | SM2019-99-4 |
| CIDD-0150169 | 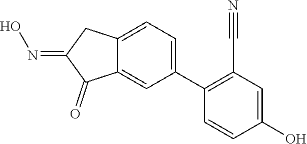 [SMILES: N#Cc1cc(O)ccc1-c1ccc2c(c1)C(=O)/C(=N/O)C2] | 5-hydroxy-2- [(2E)-2- (hydroxyimino)-3- oxo-2,3-dihydo- 1H-inden-5- yl]benzonitrile | 278.267 | 277.0 | SM2019-99-7 |
| CIDD-0150170 | 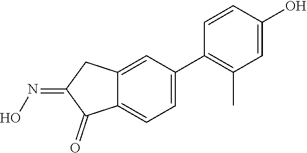 [SMILES: Cc1cc(O)ccc1-c1ccc2c(c1)C/C(=N/O)C2=O] | (2Z)-5-(4- hydroxy-2- methylphenyl)-2- (hydroxyimino)- 2,3-dihydro-1H- inden-1-one | 267.284 | 266.0 | SM2017-99-9 |
| CIDD-0150171 | 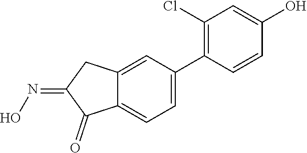 [SMILES: O=C1/C(=N\O)Cc2cc(-c3ccc(O)cc3Cl)ccc21] | (2Z)-5-(2-chloro- 4-hydroxyphenyl)- 2-(hydroxyimino)- 2,3-dihydro-1H- inden-1-one | 287.7 | 286.0 | SM2019-99-11 |
| CIDD-0150172 | 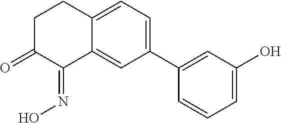 [SMILES: O=C1CCc2ccc(-c3cccc(O)c3)cc2/C1=N/O] | (1Z)-1- (hydroxyimino)-7- (3-hydroxyphenyl)- 1,2,3,4- tetrahydronapthalen- 2-one | 267.284 | 268.2 | SM2019-99-12 |
| CIDD-0150173 | 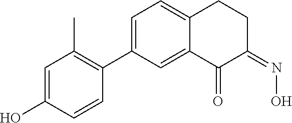 [SMILES: Cc1cc(O)ccc1-c1ccc2c(c1)C(=O)/C(=N\O)CC2] | (2Z)-7-(4-hydroxy- 2-methylphenyl)- 2-(hydroxyimino)- 1,2,3,4- tetrahydronapthalen- 1-one | 281.311 | 280.0 | SM2019-99-14-2 |
| CIDD-0150174 | 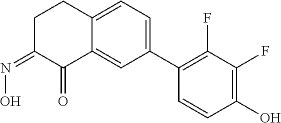 [SMILES: O=C1/C(=N\O)CCc2ccc(-c3ccc(O)c(F)c3F)cc21] | (2Z)-7-(2,3- difluoro-4- hydroxyphenyl)-2- (hydroxyimino)- 1,2,3,4- tetrahydronapthalen- 1-one | 303.265 | 304.2 | SM2019-99-14-3 |
| CIDD-0150175 | 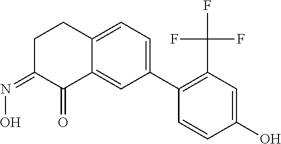 [SMILES: O=C1/C(=N\O)CCc2ccc(-c3ccc(O)cc3C(F)(F)F)cc21] | (2Z)-7-[4-hydroxy- 2- (trifluoromethyl)phen- yl]-2- (hydroxyimino)- 1,2,3,4- tetrahydronapthalen- 1- | 335.282 | 334.0 | SM2019-99-14-4 |
| CIDD-0150176 | 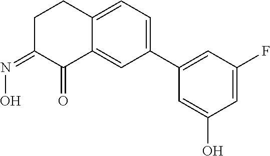 [SMILES: O=C1/C(=N\O)CCc2ccc(-c3cc(O)cc(F)c3)cc21] | (2Z)-7-(3-fluoro- 5-hydroxyphenyl)- 2-(hydroxyimino)- 1,2,3,4- tetrahydronapthalen- 1-one | 285.274 | 286.1 | SM2019-99-14-5 |
| CIDD-0150177 | 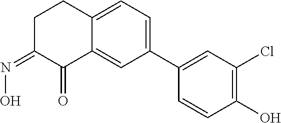 [SMILES: O=C1/C(=N\O)CCc2ccc(-c3ccc(O)c(Cl)c3)cc21] | (2Z)-7-(3-chloro- 4-hydroxyphenyl)- 2-(hydroxyimino)- 1,2,3,4- tetrahydronapthalen- 1-one | 301.73 | 302.1 | SM2019-99-14-6 |
| CIDD-0150178 | 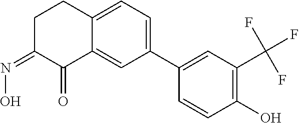 [SMILES: O=C1/C(=N\O)CCc2ccc(-c3ccc(O)c(C(F)(F)F)c3)cc21] | (2Z)-7-[4-hydroxy- 3- (trifluoromethyl)phen- yl]-2-(hydroxyimino)- 1,2,3,4- tetrahydronapthalen- 1- | 335.282 | 333.9 | SM2019-99-14-7 |
| CIDD-0150179 | 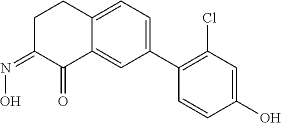 [SMILES: O=C1/C(=N\O)CCc2ccc(-c3ccc(O)cc3Cl)cc21] | (2Z)-7-(2-chloro-4- hydroxyphenyl)-2- (hydroxyimino)- 1,2,3,4- tetrahydronapthalen- 1-one | 301.73 | 302.0 | SM2019-99-14-8 |
| CIDD-0150180 | 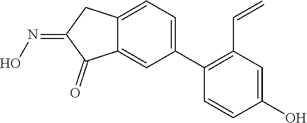 [SMILES: C=Cc1cc(O)ccc1-c1ccc2c(c1)C(=O)/C(=N\O)C2] | (2Z)-6-(2-ethenyl- 4-hydroxyphenyl)- 2-(hydroxyimino)- 2,3-dihydro-1H- inden-1-one | 279.295 | 278.0 | SM2019-99-15 |
| CIDD-0150184 | 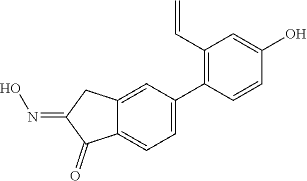 [SMILES: C=Cc1cc(O)ccc1-c1ccc2c(c1)C/C(=N\O)C2=O] | (2E)-5-(2-ethenyl- 4-hydroxyphenyl)- 2-(hydroxyimino)- 2,3-dihydro-1H- inden-1-one | 279.295 | 280.1 | SM2019-99-16 |
| CIDD-0150645 | 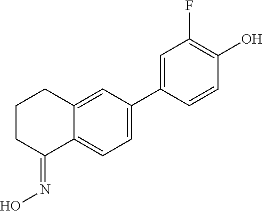 [SMILES: O/N=C1\CCCc2cc(-c3ccc(O)c(F)c3)ccc21] | 2-fluoro-4-[(5E)- 5-(hydroxyimino)- 5,6,7,8- tetrahydronapthalen- 2-yl]phenol | 271.291 | 272.3 | SM2019-99-1 |
| CIDD-0150646 | 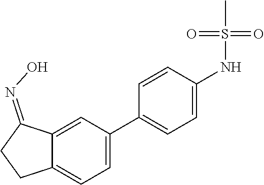 [SMILES: CS(=O)(=O)Nc1ccc(-c2ccc3c(c2)/C(=N\O)CC3)cc1] | N-(4-[(3Z)-3- (hydroxyimino)- 2,3-dihydro-1H- inden-5- yl]phenyl)methane- sulfonamide | 316.38 | 317.2 | SM2019-99-76 |
| CIDD-0150647 | 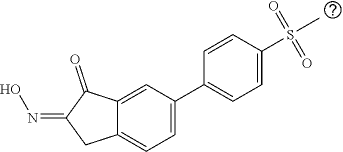 [SMILES: CS(=O)(=O)c1ccc(-c2ccc3c(c2)C(=O)/C(=N\O)C3)cc1] | 4-[(2Z)-2- (hydroxyimino)- 3-oxo-2,3-dihydro- 1H-inden-5- yl]benzene-1- sulfonamide | 316.33 | 314.8 | SM2019-99-78 |
| CIDD-0150648 | 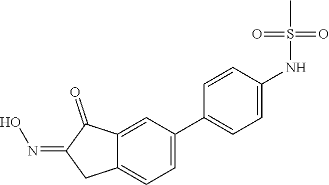 [SMILES: CS(=O)(=O)Nc1ccc(-c2ccc3c(c2)C(=O)/C(=N\O)C3)cc1] | N-(4-[(2Z)-2- (hydroxyimino)-3- oxo-2,3-dihydro- 1H-inden-5- yl]phenyl)methane- sulfonamide | 330.36 | 328.8 | SM2019-99-82 |
| CIDD-0150649 | 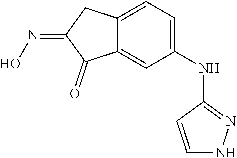 [SMILES: O=C1/C(=N\O)Cc2ccc(Nc3cc[nH]n3)cc21] | (2Z)-2- (hydroxyimino)-6- [(1H-pyrazol-3- yl)amino]-2,3- dihydro-1H-inden- 1-one | 242.238 | 243.2 | SM2019-99-93 |

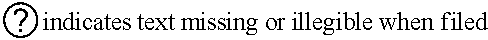

1. A compound of Formula I: 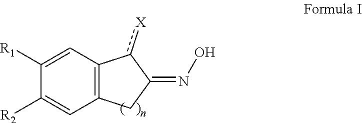
wherein,
R₁ and R₂ are independently hydrogen, alkyl, branched alkyl, substituted alkyl, aryl, substituted aryl, —CH₂-Aryl, or —CH₂-substituted aryl;
X is selected from O, —OH, N—OH, Hz, —O—R₄, bonded by a single bond or double bond, wherein R₄ is selected from hydrogen, alkyl, branched alkyl, heteroalkyl, aryl, heteroaryl, —CH₂-aryl, or —CH₂-heteroaryl; and n is 1 or 2.

2. The compound of claim 1, wherein the aryl is fused aryl, heterocyclic, or heteroaryl.

3. The compound of claim 2, wherein the aryl is a substituted aryl.

4. The compound of claim 3, wherein the substituted aryl is a substituted benzene.

5. The compound of claim 3, wherein the substituted aryl is a substituted heterocycle.

6. The compound of claim 3, wherein the substituted aryl is a substituted heteroaryl.

7. The compound of any one of claim 3 to 6, wherein the aryl comprises 1, 2, 3, 4 or 5 substituent selected from halogen, —OH, —O—R₅, —NO₂, CO₂H, —CN, —CF₃, CO₂—R₅, CONH₂, CONHR₅, CON(R₅)₂, —NH₂, —NHR₅, —N(R₅)₂, —NHC(O)R₅, alkyl, branched alkyl, substituted alkyl, halogenated methyl, or halogenated O-alkyl, wherein R₅ is selected from hydrogen, alkyl, branched alkyl, heteroalkyl, aryl, heteroaryl, —CH₂-aryl, or —CH₂-heteroaryl.

8. The compound of claim 7, wherein the halogenated methyl is mono-halogen substituted methyl, di-halogen substituted methyl, or tri-halogen substituted methyl.

8. The compound of claim 8 or claim 9, wherein the halogen flourine.

9. The compound of claim 7, wherein the halogenated O-alkyl is a mono-halogen substituted alkyl, di-halogen substituted alkyl, or tri-halogen substituted alkyl.

9. The compound of any one of claims 1 to 8, wherein n is 1.

10. The compound of any one of claims 1 to 9, wherein n is 2.

11. The compound of claim 1, wherein the compound is selected from (2Z)-6-(3-chloro-4-hydroxyphenyl)-2-(hydroxyimino)-2,3-dihydro-1H-inden-1-one; (2Z)-6-[4-hydroxy-3-(trifluoromethyl)phenyl]-2-(hydroxyimino)-2,3-dihydro-1H-inden-1-one; 2-hydroxy-5-[(2Z)-2-(hydroxyimino)-3-oxo-2,3-dihydro-1H-inden-5-yl]benzonitrile; (2Z)-2-(hydroxyimino)-6-(4-hydroxyphenyl)-2,3-dihydro-1H-inden-1-one; (2Z)-2-(hydroxyimino)-6-(4-hydroxyphenyl)-2,3-dihydro-1H-inden-1-one; (2Z)-2-(hydroxyimino)-6-(1-methyl-1H-indazol-6-yl)-2,3-dihydro-1H-inden-1-one; (2Z)-6-(3-chloro-4-hydroxyphenyl)-2-(hydroxyimino)-2,3-dihydro-1H-inden-1-one; (2Z)-6-(3-chloro-4-hydroxyphenyl)-2-(hydroxyimino)-2,3-dihydro-1H-inden-1-one; 4-[(2Z)-2-(hydroxyimino)-2,3-dihydro-1H-inden-5-yl]-2-(trifluoromethyl)phenol; 2-chloro-4-[(2Z)-2-(hydroxyimino)-2,3-dihydro-1H-inden-5-yl]phenol; (2Z)-6-(3-fluoro-5-hydroxyphenyl)-2-(hydroxyimino)-2,3-dihydro-1H-inden-1-one; (2E)-6-(3-fluoro-4-hydroxyphenyl)-2-(hydroxyimino)-2,3-dihydro-1H-inden-1-ol; 2-fluoro-4-[3-(hydroxyimino)-2,3-dihydro-1H-inden-5-yl]phenol; 2-fluoro-4-[(3E)-3-(hydroxyimino)-2,3-dihydro-1H-inden-5-yl]phenol; (2Z)-2-(hydroxyimino)-6-(1H-indazol-6-yl)-2,3-dihydro-1H-inden-1-one; 2-fluoro-4-[(2Z)-2-(hydroxyimino)-2,3-dihydro-1H-inden-5-yl]phenol; 2-hydroxy-5-[(2E)-2-(hydroxyimino)-1-oxo-2,3-dihydro-1H-inden-5-yl]benzonitrile; (2E)-2-(hydroxyimino)-5-(4-hydroxyphenyl)-2,3-dihydro-1H-inden-1-one; (2E)-5-(3-fluoro-4-hydroxyphenyl)-2-(hydroxyimino)-2,3-dihydro-1H-inden-1-one; (2E)-5-(3-chloro-4-hydroxyphenyl)-2-(hydroxyimino)-2,3-dihydro-1H-inden-1-one; (2E)-5-[4-hydroxy-3-(trifluoromethyl)phenyl]-2-(hydroxyimino)-2,3-dihydro-1H-inden-1-one; (2E)-5-(3-fluoro-5-hydroxyphenyl)-2-(hydroxyimino)-2,3-dihydro-1H-inden-1-one; 4-[(1E)-1-(hydroxyimino)-2,3-dihydro-1H-inden-5-yl]-2-(trifluoromethyl)phenol; (2E)-5-(3-fluoro-5-hydroxyphenyl)-2-(hydroxyimino)-2,3-dihydro-1H-inden-1-ol; (2E)-6-(3-chloro-4-hydroxyphenyl)-2-(hydroxyimino)-1,2,3,4-tetrahydronaphthalen-1-one; 2-chloro-4-[(5E)-5-(hydroxyimino)-5,6,7,8-tetrahydronaphthalen-2-yl]phenol; (2Z)-6-(4-hydroxy-3-methylphenyl)-2-(hydroxyimino)-2,3-dihydro-1H-inden-1-one; (2Z)-6-(4-hydroxy-3-methoxyphenyl)-2-(hydroxyimino)-2,3-dihydro-1H-inden-1-one; (2Z)-6-(4-hydroxy-3,5-dimethylphenyl)-2-(hydroxyimino)-2,3-dihydro-1H-inden-1-one; (2Z)-6-(2H-1,3-benzodioxol-5-yl)-2-(hydroxyimino)-2,3-dihydro-1H-inden-1-one; (2Z)-2-(hydroxyimino)-6-(3-hydroxyphenyl)-2,3-dihydro-1H-inden-1-one; (2E)-2-(hydroxyimino)-6-[4-(2H-1,2,3,4-tetrazol-5-yl)phenyl]-2,3-dihydro-1H-inden-1-one; (2Z)-6-[4-hydroxy-2-(trifluoromethyl)phenyl]-2-(hydroxyimino)-2,3-dihydro-1H-inden-1-one; (2Z)-6-(2-chloro-4-hydroxyphenyl)-2-(hydroxyimino)-2,3-dihydro-1H-inden-1-one; (2Z)-6-(4-hydroxy-2-methylphenyl)-2-(hydroxyimino)-2,3-dihydro-1H-inden-1-one; (2Z)-2-(hydroxyimino)-6-(2-methoxypyrimidin-5-yl)-2,3-dihydro-1H-inden-1-one; (2Z)-6-(2,4-dimethoxyphenyl)-2-(hydroxyimino)-2,3-dihydro-1H-inden-1-one; (2Z)-6-(2-fluoro-4-hydroxyphenyl)-2-(hydroxyimino)-2,3-dihydro-1H-inden-1-one; (2Z)-6-(2,3-difluoro-4-hydroxyphenyl)-2-(hydroxyimino)-2,3-dihydro-1H-inden-1-one; (2Z)-6-(4-hydroxy-2-methylphenyl)-2-(hydroxyimino)-1,2,3,4-tetrahydronaphthalen-1-one; 4-[(5E)-5-(hydroxyimino)-5,6,7,8-tetrahydronaphthalen-2-yl]-3-methylphenol; or (2Z)-2-(hydroxyimino)-6-(2-hydroxypyrimidin-5-yl)-2,3-dihydro-1H-inden-1-one.

12. The compound of claim 1, wherein the compound is (2E)-5-(2-ethenyl-4-hydroxyphenyl)-2-(hydroxyimino)-2,3-dihydro-1H-inden-1-one or (2Z)-6-(3-fluoro-4-hydroxyphenyl)-2-(hydroxyimino)-2,3-dihydro-1H-inden-1-one.

13. A method of treating cancer comprising administering an effective amount of a compound of any one of claim 1 to 12.

14. A method of providing neuroprotection comprising administering an effective amount of a compound of any one of claim 1 to 12.

15. A method of agonizing an Estrogen Receptor Beta comprising administering an effective amount of a compound of any one of claim 1 to 12.
